(19) United States
(12) Patent Application Publication
(10) Pub. No.: US 2020/0054343 A1
(43) Pub. Date: Feb. 20, 2020

(54) INFLATABLE ANCHORING STRUCTURE FOR IMPLANT AND DELIVERY SYSTEM

(71) Applicant: Cornell University, Ithaca, NY (US)

(72) Inventors: James K. MIN, Brooklyn, NY (US); Bobak MOSADEGH, New York, NY (US); Tracey LUSTIG, New York, NY (US); Simon DUNHAM, New York, NY (US)

(21) Appl. No.: 16/603,514

(22) PCT Filed: Apr. 6, 2018
(86) PCT No.: PCT/US2018/026532

§ 371 Date: Oct. 7, 2019

Related U.S. Application Data
(60) Provisional application No. 62/483,095, filed on Apr. 7, 2017.

(51) Int. Cl. A61B 17/12

(52) U.S. Cl. CPC A61B 17/12136; A61B 2017/00867; A61B 17/12122

(57) ABSTRACT

Systems and methods for occluding a body cavity including providing an implantable void-filling device, wherein the implantable void-filling device includes an inflatable implant that defines an interior of the implantable void-filling device. The inflatable implant is capable of being filled with an inflation material to cause the inflatable implant to expand from a collapsed configuration to an expanded configuration. The implantable void-filling device also includes a connection hub attached to an exterior surface of the inflatable implant and a plurality of independent anchors coupled to and extending out from the connection hub along the length of the inflatable implant, such that the plurality of anchors collectively surround the inflatable implant. The connection hub and anchors are configured such that expansion of the anchors away from the surface of the inflatable implant anchors the void filling device to a body tissue.

### CROSS-REFERENCE TO RELATED APPLICATIONS

The present application claims priority to, and the benefit of, U.S. Provisional Patent Application No. 62/483,095 entitled “INFLATABLE ANCHORING STRUCTURE FOR IMPLANT AND DELIVERY SYSTEM” and filed on Apr. 7, 2017, the entire contents of which are hereby incorporated by reference for all purposes.

### BACKGROUND OF THE INVENTION

The disclosure herein relates to inflatable structures and methods and delivery devices therefore that conform to the native structure of the target location, such as a cavity, void, defect, or connection between body structures.

### SUMMARY OF THE INVENTION

According to one aspect of the disclosure, a system for occluding a body cavity includes an implantable void-filling device, wherein the implantable void-filling device includes an inflatable implant defining an interior of the implantable void-filling device. The inflatable implant is capable of being filled with an inflation material to cause the inflatable implant to expand from a collapsed configuration to an expanded configuration. The implantable void-filling device also includes a connection hub attached to an exterior surface of the inflatable implant, and a plurality of independent anchors coupled to and extending out from the connection hub along the length of the inflatable implant, such that the plurality of anchors collectively surround the inflatable implant. The connection hub and anchors are configured such that expansion of the anchors away from the surface of the inflatable implant anchors the void filling device to a body tissue. In some implementations, the system also includes a coating configured to cover the connection hub and at least a portion of the anchors. In some implementations, the coating includes a fabric covering attached to the connection hub and at least a portion of the independent anchors.

In some implementations, the inflatable implant includes an ultra-compliant balloon configured to conform to the individual shape of the body cavity. In some implementations, the inflation material includes a gas, a liquid, or a gel. In some implementations, the ultra-compliant balloon includes of an elastomer, a polymer, or a fabric reinforced polymer.

In certain implementations, the inflatable implant includes an over-sized balloon configured to conform to the individual shape of the body cavity. In some implementations, the inflatable implant is configured to conform to the individual shape of the body cavity at substantially zero pressure.

In some such implementations, the system also includes a catheter assembly configured to deliver the implantable void-filling device to the body cavity. The catheter assembly of the system may include a handle including an inflation port attachable to an inflation fluid cartridge containing inflation fluid, and an inner shaft slideably contained within an outer shaft and defining an inflation lumen in fluid communication with the inflation port and the inflatable implant, wherein a connection assembly removably joins the inflatable implant and the catheter assembly. In certain implementations, the system further includes a release mechanism configured to release the inner shaft of the catheter assembly through the access lumen of the septum without causing proximal retraction of the inflatable implant.

In some implementations, the plurality of anchors include tines arranged along the lengths of the respective anchors and are configured to, upon exposure to an activation energy, curve outward from the respective anchors to engage a body tissue. In some implementations, the plurality of anchors include of a shape-memory alloy. In some implementations, the curved anchors have a straightened delivery configuration and an expanded configuration, wherein the anchors assume the expanded configuration upon exiting the outer shaft whereby the anchors passively or actively curve outwardly upon exposure to an activation energy. In some implementations, the curved anchors have a straightened delivery configuration and an expanded configuration, wherein the anchors assume the expanded configuration upon exiting the outer shaft because they are released from a compressed state.

According to another aspect of the disclosure, a method for occluding a body cavity includes providing an implantable void-filling device. The implantable void-filling device includes an inflatable implant defining an interior of the implantable void-filling device, wherein the inflatable implant is capable of being filled with an inflation material to cause the inflatable implant to expand from a collapsed configuration to an expanded configuration. The implantable void-filling device also includes a connection hub attached to an exterior surface of the inflatable implant, and a plurality of independent anchors extending coupled to and extending out from the connection hub along the length of the inflatable implant, such that the plurality of anchors collectively surround the inflatable implant. The connection hub and anchors are configured such that expansion of the anchors away from the surface of the inflatable implant anchors the void filling device to a body tissue.

The method also includes providing a catheter assembly configured to deliver the implantable void-filling device to the body cavity. The method also includes navigating the implantable void-filling device to a target body cavity with the catheter assembly. In some implementations, navigating an inflatable implant to a target body cavity with the catheter assembly includes navigating the catheter assembly having said inflatable implant stored in a distal end thereof.

The method also includes inserting the implantable void-filling device in the target body cavity. In some implementations, inserting the inflatable implant in the target body cavity includes ejecting the inflatable implant from within a distal end of the catheter assembly. The method also includes filling the inflatable implant with an inflation material through an inner shaft contained within the catheter assembly and anchoring the implantable void-filling device to the inside of the body cavity through the plurality of independent anchors. In some implementations, filling the inflatable implant with an inflation material includes filling the inflatable implant with a plurality of fluids, gases, or curing liquids that interact with each other. In some implementations, the inflatable implant includes an ultra-compliant balloon configured to conform to the individual shape of the body cavity. In some implementations, the ultra-compliant balloon includes of an elastomer, a polymer, or a fabric reinforced polymer.

The method also includes releasing the inflatable implant from the catheter assembly and retracting the delivery device from the body tissue. In some implementations, releasing the inflatable implant from the catheter assembly includes rotating a component of the catheter assembly relative to a mating component of the void-filling device, such that the two components disengage each other. In some implementations, anchoring the implantable void-filling device to the inside of the body cavity includes engaging the body tissue with tines arranged along the lengths of the respective anchors that are configured to, upon exposure to an activation energy, curve outward from the respective anchors to engage a body tissue. In some implementations, the plurality of anchors include a shape memory alloy nitinol.

### BRIEF DESCRIPTION OF THE DRAWINGS

The skilled artisan will understand that the figures, described herein, are for illustrative purposes only. It is to be understood that in some instances various aspects of the described implementations may be shown exaggerated or enlarged to facilitate an understanding of the described implementations. In the drawings, like reference characters generally refer to like features, functionally similar, and/or structurally similar elements throughout the various drawings. The drawings are not necessarily to scale, emphasis instead being placed upon illustrating the principles of the teachings. The drawings are not intended to limit the scope of the present teachings in any way. The systems and method disclosed herein may be better understood from the following illustrative description with reference to the following drawings in which:

FIG. 1 shows a schematic diagram of an example implantable void-filling device and catheter assembly.

FIG. 2 illustrates an example implementation of an implantable void-filling device as shown in FIG. 1.

FIG. 3 illustrates an example implementation of an implantable void filling device where anchors surround the inflatable implant, such that the inflatable implant expands around the anchors.

FIGS. 4A-4C show three different balloon molds that can be used to create an inflatable implant for the void-filling devices shown in FIGS. 1-3.

FIGS. 5A-5C show three example implementation of different anchor and tine configurations suitable for use with the void-filling devices shown in FIGS. 1-3.

FIGS. 6A-6B illustrate example implementations of the independent anchors and corresponding tines suitable for use with the void-filling devices shown in FIGS. 1-3.

FIG. 7 illustrates an example implementation of a plurality of anchors in a bent configuration and attached to a connection hub.

FIG. 8 shows an example implementation of a coating attached to the exterior proximal portion of an implantable void-filling device suitable for use with the void-filling devices shown in FIGS. 1-3.

FIG. 9 illustrates an example implementation of a catheter assembly attached to an implantable void-filling device suitable for use with the void-filling devices shown in FIGS. 1-3.

FIG. 10 illustrates an example implementation of a connection assembly between a catheter assembly and an implantable void-filling device.

FIG. 11 illustrates an example method 1100 for delivering the implantable void-filling device into a subject's body cavity.

FIG. 12 illustrates an example implementation of a catheter assembly delivering an implantable void-filling device to a LAA.

### DESCRIPTION OF IMPLEMENTATIONS

Specific implementations of the invention will now be described with reference to the accompanying drawings. This invention may, however, be embodied in many different forms and should not be construed as limited to the implementations set forth herein; rather, these implementations are provided so that this disclosure will be thorough and complete, and will fully convey the scope of the invention to those skilled in the art. The terminology used in the detailed description of the implementations illustrated in the accompanying drawings is not intended to be limiting of the invention. The examples of specific implementations are provided primarily for illustrative purposes.

Interventional medicine often calls for the implantation of a prosthetic device that occludes a native void or defect, or provides an anchored platform for another prosthesis. Examples of voids or defects that sometime require filling include the left atrial appendage, (“LAA”), septal defects, aneurysms, pseudo-aneurysms, and colonic outpouchings. Examples of prosthetic devices that require anchoring include both prosthetic devices that fill a native void or defect, as well as prosthetic valves that replace defective or damaged valves in the body.

The LAA is an example appendage that can promote stagnant blood flow, which results in thrombus formation. In patients suffering from atrial fibrillation, it is thought that the LAA is a prominent source of thrombus formation. In such patients, the risk of thrombus formation in voids, such as the LAA, can be reduced, if not eliminated, by reducing the effective volume of the LAA where blood can pool and stagnate. Previous efforts to reduce the volume of the LAA have included either obstructing the LAA by placing stent devices that anchor into the proximal portion of the appendage (without filling the LAA body), excising the tissue wall forming the LAA, or structurally modifying the LAA by fixing the wall of the LAA in a collapsed state.

Of these solutions, placing a stent at the level of the ostium of the LAA is thought to be the least risky procedure because it does not involve puncturing, cutting, or removing cardiac tissue. However, the risks involved with placing a stent at the level of the ostium of the LAA without occluding the cavity include the device becoming dislodged, interfering with normal blood flow through the left atrium, traumatizing the tissue walls, causing pericardial effusion and complete filling of the LAA which may increase the likelihood of residual thrombus formation. Other aneurysms, pseudo-aneurysms, and outpouchings present similar hazards and challenges. The systems and methods describe herein provide a device for complete sealing of a void or defect, such as the LAA, in a patient that gently conforms to the anatomy of the target site.

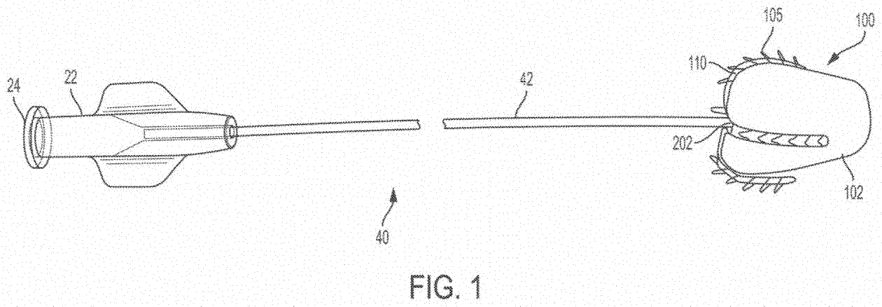

FIG. 1 shows a schematic diagram of an example implantable void-filling device and catheter assembly. The system includes an implantable void-filling device 100 and a catheter assembly 40. The implantable void-filling device 100 includes an inflatable implant 102, such as a balloon, a plurality of independent anchors 110, and a connection hub 202. The plurality of independent anchors 110 also includes a plurality of tines 105 arranged along the length of each respective anchor 110. Upon exposure of an activation energy, the tines 105 curve outward from the respective anchor to engage a body tissue. In some implementations, the anchors and the tines are manufactured from a shape memory alloy, such as nitinol, wherein a subject's body temperature acts as an activation energy to change the shape of the anchors 110 and tines 105 after delivery into the body cavity. In some implementations, the tines 105 curve outward after being released from a compressed state in the catheter assembly 40. The plurality of anchor legs 110 and tines 105 attach the implant into the subject's body tissue upon implantation.

FIG. 1 also includes a catheter assembly 40. Generally, the catheter assembly 40 accesses and deploys the implantable void-filling device 100 to the target anatomy. The catheter assembly 40 may include one or more ports 24, a handle 22, and an outer shaft 42. In some implementations, the catheter assembly 40 may also allow for simultaneous or sequential injection or removal of injected materials into the inflatable implant 102 by means of the ports 24 located on the catheter handle 22. This mechanism may include, but is not limited to, one or more lumens for injection or withdrawal of injection fluid; and/or one or more ports 24 that can serve the purposes of filling, removing, venting, insufflation, visualization or administration of one or a plurality of materials. In some implementations, filling of the void-filling implant 100 by means of the catheter assembly 40 may include the simultaneous delivery of two or more substances through multiple ports 24, or sequential delivery of two or more substances, wherein the substances interact or co-exist with each other. The filling of the void-filling implant 100 may also include a rate-based delivery where the implant is filled based on the known volume of the body cavity and inflation is conducted in a controlled fashion by quantitative or semi-quantitative volume based on pre-procedural or real-time 2-dimensional or 3-dimensional imaging. Also, the void-filling implant may include image-based filling such that real-time imaging monitors the size of the implant.

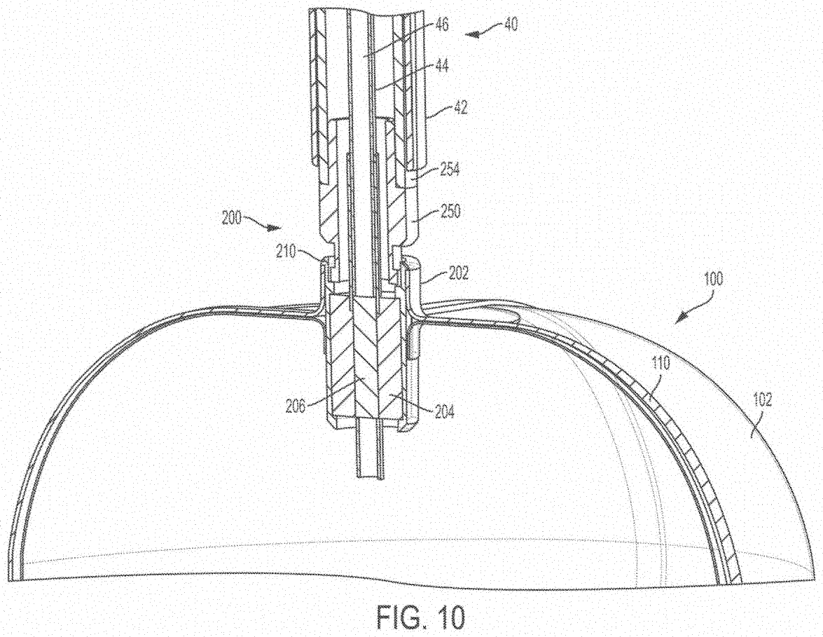

The catheter assembly 40 can also include a release mechanism useable to release the void-filling implant 100 from the catheter assembly 40. The configuration of the release mechanism is dependent on the configuration of a connection assembly (not shown), discussed in more detail below in relation to FIG. 10. For example, if the connection assembly includes a rotating disengagement, the release mechanism will includes a knob or other rotating feature that allows the user to rotate the catheter assembly with respect to implant 100. In another example, if the connection assembly includes a sliding or otherwise axial disengagement configuration, the release mechanism may include a push button or trigger mechanism that allows a user to retract or extend one component relative to another.

In some implementations, the catheter assembly 40 may also include a locking mechanism, a distal rigid or semi-rigid rim, a mechanism for allowing rotational movement of the implant, and a method for remotely actuating the locking mechanism. The catheter assembly 40 may be distally associated with a locking mechanism or connection assembly that allows for device retention and manipulation before, during, or after injection of the material into the inflatable structure. The catheter assembly 40 may further possess an outer sheath 42 having a distal rigid or semi-rigid edge that enhances retraction or delivery of the implant. The catheter assembly 40 may also include a mechanism that allows for remote actuation of the locking mechanism or connection assembly, such as by torqueing or rotating the catheter, or axial manipulation of a pusher catheter within the outer sheath 42. The delivery catheter may further possess an ability or means that allows for finite or continuous controlled retraction of an implant, for example by retraction of the outer sheath 42.

The locking mechanism, the rim, and the mechanisms for rotational movement and actuating the locking mechanism are discussed in further detail in relation to FIG. 10 below.

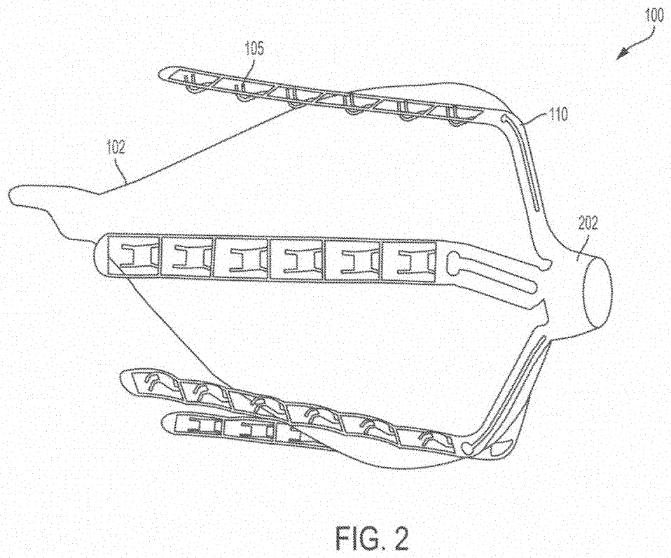

FIG. 2 illustrates an example implementation of an implantable void-filling device 100 as shown in FIG. 1. As mentioned above, the implantable void-filling device 100 incudes a connection hub 202, an inflatable implant 102, and a plurality of independent anchors 110.

In general, the inflatable implant 100 (also referred to as a balloon) is a structure that provides or possesses an adaptive anchor to the anatomy of a native tissue cavity, void, defect or connection between two body structures. The inflatable implant 102 adapts or is shaped in a manner that does not disturb or damage the native tissue. The inflatable implant 102 provides an adaptive seal to the native anatomy. In some implementations, the inflatable implant 102 may be configured such even when inflated sufficiently to fill a target body cavity, the fluid filling the inflatable implant exerts substantially zero-stress against the implant's walls to ensure that the inflatable implant or balloon 102 does not further expand into portions of the body other than the targeted body cavity. In some implementations, in order to achieve a substantially zero-stress post-inflation configuration, the inflatable implant 102 may be inflated to a substantially zero-pressure. In some implementations, in order to achieve a substantially zero-stress post-inflation configuration the balloon may be manufactured from an ultra-compliant material configured to conform to the individual shape of the body cavity. For example, the ultra-compliant balloon may be manufactured from silicones, polyurethanes, hydrogels, or any polymer with extensibility over 30% and Young's modulus less than 100 MPa. In other implementations, the inflatable implant 102 may be over-sized to achieve a substantially zero-pressure post-inflation configuration. In some implementations, the use of a very conformable and/or ultra-compliant balloon as the inflatable implant 102 provides the additional benefit of allowing a physician to obtain an improved image of the shape of the space the implant is filling. For example, the balloon may be filled with a radiopaque or echogenic substance and as the balloon fills, it expands into all the different crevices of the body cavity, which allows the physician to obtain an accurate image of the patient's body cavity. In some implementations, for example, the inflatable void-filling device may be used to occlude the LAA within a subject's atrium. The pressure in the atrium of the heart is very small, therefore, a filled inflatable implant maintained at a positive (substantially non-zero) pressure will have the tendency to expand out of the proximal end of the atrial appendage into the atrium after inflation; however, an ultra-thin and compliant or over-sized inflatable implant having a substantially zero pressure post-inflation configuration will occlude the LAA without the same tendency to expand into the lower pressure atrium, potentially obstructing undesired portions of the heart's left atrium. As used herein a substantially zero pressure refers to a pressure between 0 and about 30 mm Hg.

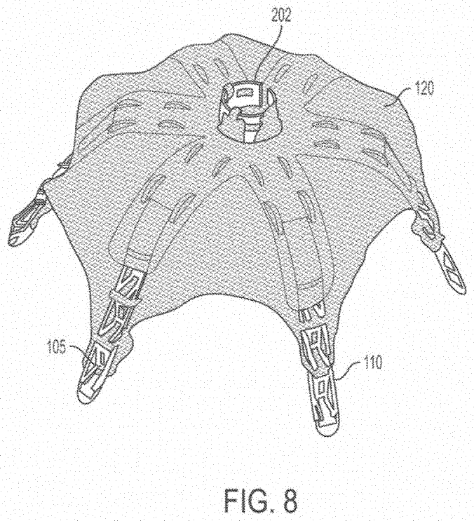

In other implementation, a balloon filled to a greater pressure can be employed so long as it is positioned within the plurality of independent anchors 110 and/or an exterior coating (see, e.g. FIG. 8) that prevents the balloon from expanding into the left atrium. In such implementations, a balloon manufactured from stiffer materials that can achieve conformability at higher pressures can be employed, as long as the balloon is not filled to a pressure that damages the surrounding body tissue.

In some implementations, the inflatable implant 102 may, upon inflation, include a flat proximal top for one or a plurality of desired effects, such as to reduce or prevent thrombus in the region of the implant that is exposed to blood. In some implementations, the implant may have radiopaque or echogenic properties. Additionally, the implant 100 may possess one or more ports or accesses that can be used for filling, removing, venting, insufflation, visualization or administration of one or a plurality of materials.

In some implementations, the material used to fill the inflatable implant 102 may maintain a constant consistency and volume or it may solidify after delivery. Alternatively, the fluid may be absorbed by the body over time. Non-limiting examples of the inflation fluid include liquid-based substances such as saline, iodine or gadolinium-based contrast agents; curable polymers, curable hydrogels, silicone or foam. In some implementations, the inflation fluid may include materials capable of phase change. The change may be induced by gradual chemical reactions to a stimulus-triggered event. Examples of such events include exposure to a predetermined pH, electrochemical activation, ultrasonically induced mixing, optical methods, or inductive methods.

Referring back to FIG. 2, the inflatable implant 102 may be associated with an independent anchor structure 110 that is connected to the inflatable implant 102 by means of a connection hub 202 for preventing the balloon from expanding into the atrium. The anchors 110 may be self-deploying or actively deployed against, into or on top of the native tissue, which also ensures that the balloon will not expand into the atrium after delivery.

The plurality of independent adaptive anchors 110 can independently move with respect to the individual morphology of the patient. Each independent anchor acts independently from the other anchors connected to the connection hub 202. For example, the anchors 110 may be deployed in a fashion that may render it symmetric or asymmetric; regular or irregular; or aligned or misaligned with the native anatomy. The anchors 110 can by actively or passively re-oriented in a manner to better conform to the native anatomy. The anchors 110 may be flexible or rigid and may be associated with a single or plurality of links or segments. The anchors 110 or components of the anchors 110 may vary in shape or size along its length to adapt to the native anatomy for better anchoring. The anchors 110 may be incorporated or directly attached to the inflatable structure, or affixed to an independent retention structure, such as the connection hub. The plurality of independent anchors 110 also include a plurality of tines 105 arranged along the lengths of the respective anchors 110 and configured to, upon exposure to the activation energy, curve outward from the respective anchors 110 to engage a body tissue.

In some implementation the anchors may be shaped to give the implant 100 an apple shape, wherein the connection hub is depressed within the proximal end of the inflatable implant 102, which prevents the connection hub 202 from protruding from the implant 100. Such a configuration may be advantageous when used to occlude a void such as the LAA because the inflation port of the inflatable implant 102 remains contained within the void. In this and other implementations, the independent anchors 110 are preferably formed out of nitinol, or a similar shape memory alloy, so that the anchors 110 may be collapsed into a straight configuration during delivery through the catheter, and then assume a desired shape when released from the catheter assembly 40.

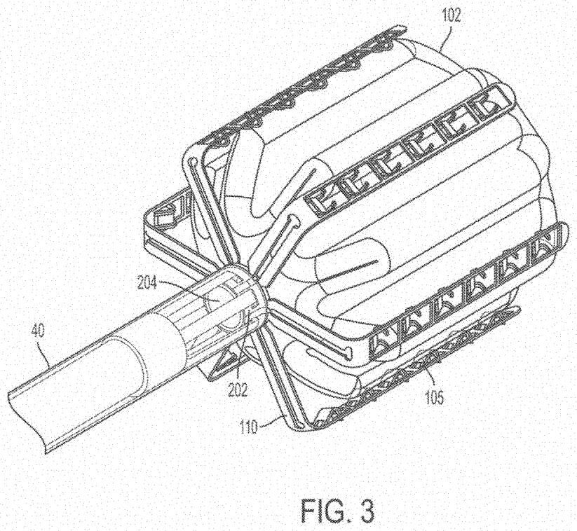

FIG. 3 illustrates an example implementation of an implantable void filling device where anchors surround an inflatable implant 102, and the inflatable implant inflates around the anchors 110. FIG. 3 shows an inflatable implant 102, a plurality of independent anchors 110, a connection hub 202, and a catheter assembly 40. The void-filling implant 100 of this implementation includes six independent anchors 110 that are adhered, integrally formed with, or are fastened to the connection hub 202. In some implementations, the inflatable implant 102 is a substantially zero pressure filled implant, as described in FIGS. 1-2, that conforms around the independent anchors 110 to further occlude the subject's body cavity without applying force sufficient to change the shape of the anchors 110. The implementation of FIG. 3 may also prevent wrinkles from forming on the balloon's surface upon deployment, which can help reduce thrombus and promote overgrowth of cellular components.

A plurality of tines 105 arranged along the length of each independent anchor 110, as shown in FIG. 3, serve as an attachment mechanism in that, once expanded, the tines 105 prevent migration of the implant 100 out of the body cavity it is filling, even if the inflatable implant 102 were to deflate. Additionally, the tines 105 provide the option of using an absorbable inflation fluid within the inflatable implant 102. The absorbable fluid provides structure to the implant, for example, while cellular outgrowth occurs on an exterior of the inflatable implant 102. Over time, the fluid within the inflatable implant 102 could be absorbed, and the combination of the anchors 110, tines 105, and the cellular overgrowth could function to prevent ingress of blood or other fluids into the void.

In some implementation, the independent anchors 110 and the tines 105 are made of nitinol and are connected to the connection hub 202. The anchors 110 are collapsed into a deliverable (such as straight) configuration during delivery. Once released from the catheter assembly 40, the anchors and tines assume an expanded, curved configuration. In some implementations, the tines are adhered or attached to the inflatable implant 102 of the void-filling device 100. In some implementations, the expansion of the tines results in a vacuum within the interior of the inflatable implant 102. This vacuum at least assists in drawing inflation fluid into the inflatable implant 102.

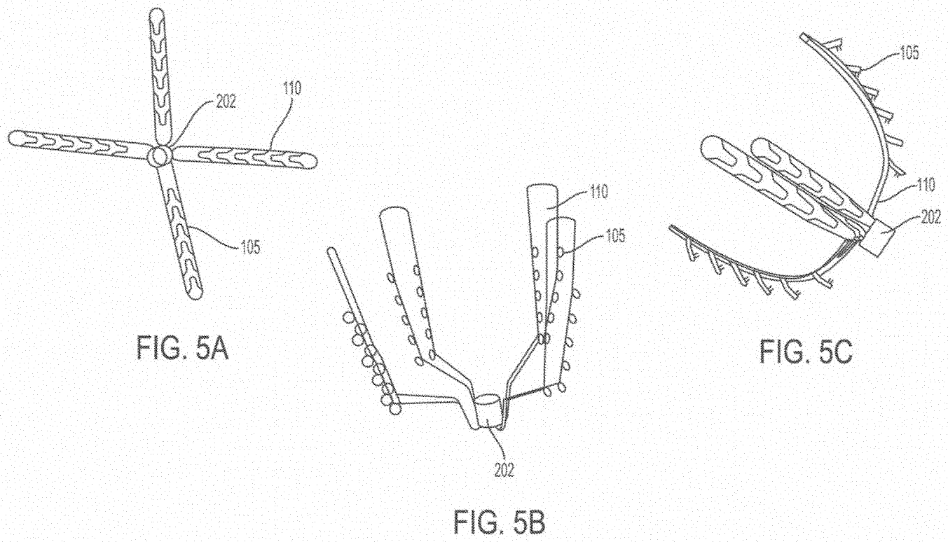

The independent anchors 110 may also serve as a location assistance device. To this end, the anchors 110 are constructed, or coated with, a radiopaque or echogenic material. Due to the curved shape of the anchors 110, a user will be able to discern, using an imaging modality such as fluoroscopy or echocardiography, the orientation of the void-filling device 100, as well as the degree to which the inflatable implant 102 is inflated. Different implementations of the tines 105 are shown in further detail in relation to FIGS. 5A-5C below.

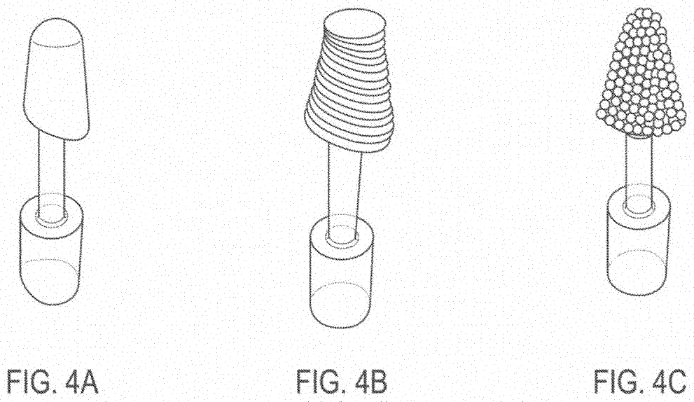

FIGS. 4A-4C show three different balloon molds that can be used to create an inflatable implant for the void-filling devices shown in FIGS. 1-3. FIGS. 4A-4C represent a variety of balloon molds that can be used in the void-filling implant. Each implementation accomplishes one or more design goals that enhance the functionality of the void-filling device for a variety of corresponding use cases depending on the geometry requirements of the particular body cavity being occluded. In FIG. 4A, the balloon mold has a smooth outer surface, which can help prevent wrinkles on the surface of the membrane after implantation. FIG. 4B has a pine tree configuration with concentric grooves along the exterior of the mold. The concentric grooves create friction between the balloon, independent anchors, and the body tissue, which helps anchor the void-filling device within the body cavity. Similarly, the mold in FIG. 4C has as a raspberry configuration, wherein the exterior of the mold has small peaks and valleys similar to a raspberry. The texture of the balloon helps anchor the inflatable implant to the body cavity. Also, the texture of FIG. 4B and FIG. 4C promote cellular overgrowth, which prevents ingress of blood or other fluids into the void.

In some implementations, undersized inflatable implants can be fabricated from an elastomer, wherein the non-limiting examples include polyurethane, silicon, or latex. In other implementations, the inflatable implant can be fabricated from non-stretchable polymers to create an over-sized balloon; such non-limiting examples of non-stretchable implants include nylon, PET, or PTFE. Also in some implementations, the void-filling device may use a fabric reinforced polymer as an inflatable implant to allow for high pressure inflation.

In some implementations, the diameter of the inflatable implant can be about 20 mm to 30 mm at the inflation size. In some implementations, the length after inflation of the inflatable implant may be about 10 mm to 30 mm.

FIGS. 5A-5C show three example anchor and tine configurations suitable for use in the void-filling device of FIGS. 1-3. FIGS. 5A-5C represent a variety of tine configurations that can be used in conjunction with independent anchors. Each tine configuration can accomplish one or more design goals that enhance the functionality of the void-filling device for a variety of corresponding use cases depending on the geometry requirements of the particular body cavity being occluded. Each implementation of FIGS. 5A-5C includes a connection hub 202, a plurality of independent anchors 110, and a plurality of tines 105 extending along the length of each independent anchor 110. FIG. 5A shows four independent anchors 110 in a relatively straight leg configuration attached to the connection hub 202 and six tines 105 along the length of each anchor 110. In some implementations, the tines 105 may include a single hook configuration, as shown in FIG. 5A. In other implementations, a single tine 105 may include two or more hooks, which will increase the attachment surface area of each anchor leg. FIG. 5B shows an example implementation of four independent anchors in a bent or expanded configuration attached to the connection hub 202 and five pairs of circular tines 105 along the length of each bent independent anchor 110. The pairs of circular tines 105 increase the surface area for a stronger attachment to the body tissue. FIG. 5C, illustrates four curved independent anchors 110 with six tines 105 extending along the length of each anchor leg 110. The tines 105 in FIG. 5C are a hook shape similar to those in FIG. 5A; however, the tines in FIG. 5C also include small barbs 106 extending from the hooks. The barbs 106 help the void-filling device attach to the subject's body tissue and they may be rounded or sharp depending on the desired level of tissue engagement.

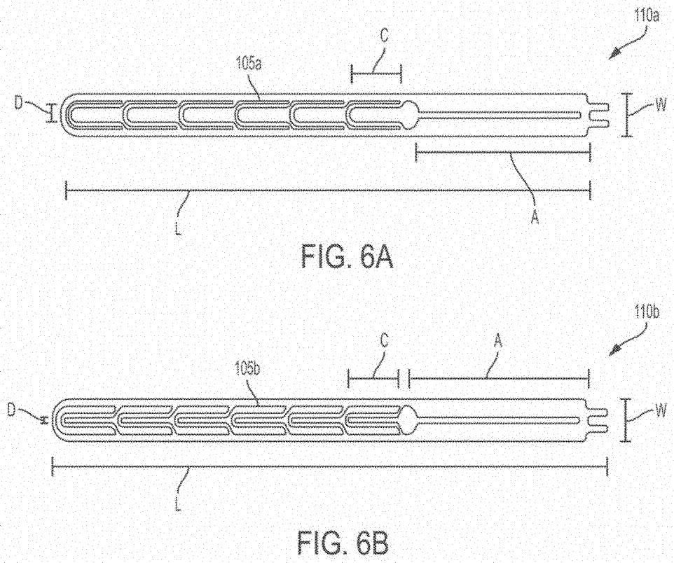

FIGS. 6A-6B illustrate example implementations of the independent anchors 110 and corresponding tines suitable for use in the void-filling device shown in FIGS. 1-3 prior to activation. Each structure accomplishes one or more design goals that enhance the functionality of the void-filling implant for a variety or corresponding use cases depending on the geometric and biophysical requirements of particular tissue cavities being occluded. As previously mentioned, the tines are arranged along the lengths of the respective anchors and configured to, upon exposure to an activation energy or after being released from a compressed state, curve outward from the respective anchors to engage a body tissue. Each of the independent anchors is proximally connected to the connection hub (not shown). Referring to FIGS. 6A and 6B, the length A represents the length of the anchor between the connection hub and the beginning of the tine series, otherwise known as the tabletop radius, which may be about 6 mm to 15 mm, and the length L of the entire anchor that includes the series of tines may be about 9 mm to 24 mm. The width W of the anchor may be about 0.5 to 2 mm, and the inner width of the tines D may be about 0.1 mm to 1 mm. The length of each tine C may be about 0.5 mm to 4 mm.

FIG. 6A illustrates a top down view of an example implementation of an independent anchor 110a. Along the length of the independent anchor are six separate tines. In FIG. 6A, the length L of the independent anchor 110a is about 32 mm, and the length C of each independent tine, for example tine 105b, is about 2.5 mm. The inner width D of each tine is about 0.5 mm. The tabletop radius A on the anchor 110a between where the anchor 110a attaches to the connection hub and the beginning of the series of tines is about 11 mm. In some implementations, the tabletop radius A on the anchor 110a between where the anchor 110a attaches to the connection hub and the beginning of the series of tines may be manufactured from a rigid material to provide a strong base for an endothelial coating and a connection for the catheter assembly. The region of the independent anchor 110a where the tines are arranged along its length may be made from a flexible material to ensure conformity with the body cavity. In some implementations, the anchors 110a are made from a single material that has varied rigidity or flexibility along its length. In some implementations, the variation is due to the anchor having a variable thickness along its length. In some implementations, the portion of the anchor 110a in which the tines are formed (the distal portion) is more flexible than the tabletop portion due to the removal or absence of material from the distal portion resulting from the space left to separate the tines from the remainder of the distal portion of the anchor 110a.

FIG. 6B, illustrates a top down view of another example implementation of an independent anchor 110b, wherein along the length of the independent anchor are six separate tines. Similar to FIG. 6A, the length L of the independent anchor 110b is about 32 mm, and the length C of each independent tine, for example tine 105b, is about 2.5 mm. The tabletop radius A on the anchor 110b between where the anchor 110b attaches to the connection hub and the beginning of the series of tines may is 11 mm, and the inner width D of each tine is about 0.3 mm. In some implementations, the length A on the anchor 110a between where the anchor attaches to the connection hub and the beginning of the series of tines may be manufactured from a rigid material to provide a strong base for the endothelial coating, and the connection for the catheter assembly. The tines arranged along the length of the anchor may be made from a flexible material to ensure conformity with the body cavity. As indicated above, in some implementations the entirety of the anchors may be formed from a single material, and the aforementioned variation in flexibility of portions of the anchor results from varying dimensions (e.g., thickness) or the introduction of structural gaps at tine locations, along the length of the anchors. In general, the dimensions and geometrics in FIGS. 6A-6B help stabilize the void-filling implant within the subject's body cavity by providing sufficient anchorage, while maintaining a size and shape to fit within a catheter assembly.

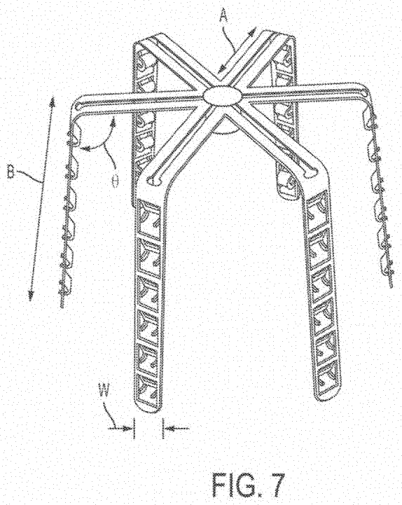

FIG. 7 illustrates an example implementation of a plurality of anchors 110 in a bent configuration, e.g., after implantation, and attached to a connection hub 202. FIG. 7 includes six independent anchors 110 attached to the connection hub 202. Also, along the length of each independent anchor 110 includes six pairs of tines 105 resembling hooks. Similar to FIGS. 6A-6B, the tabletop radius A on the anchor leg between the connection hub and the beginning of the tine series may be about 6 mm to 15 mm, and the length B of the anchor leg that includes the series of tines may be about 9 mm to 24 mm. The width W of the anchor leg may be about 0.5 to 2 mm. After implantation, the angle θ for each independent anchor between the tabletop radius A and the series of tines B may be about −60° to 120° depending on the specific morphology of the anatomy to which the anchors attach. Each independent anchors acts independently from the others; therefore, the angle θ may be different for each anchor depending on the shape of body cavity.

FIG. 8 shows an example implementation of a coating 120 attached to the exterior proximal portion of an implantable void-filling device suitable for use in devices shown in FIGS. 1-3. FIG. 8 shows the coating 120, a connection hub 202, a plurality of independent anchors 110, and a plurality of tines 105 protruding along the length of the anchors 110. In some implementations, as shown in FIG. 8, the coating is a fabric covering. The fabric covering 120 may be, e.g., manufactured from a PET textile. The covering 120 is attached to the exterior side of the independent anchors 110, which enhances endothelialization of the implant to the body tissue and reduces the risk of thrombosis. The covering 120 is attached to the connection hub 202 and the independent anchors 110 without interfering with the functionality of the anchors 110 or the tines 105; therefore, the tines 105 and the anchors 110 conserve the ability to independently attach and stabilize the void-filling implant within the body cavity.

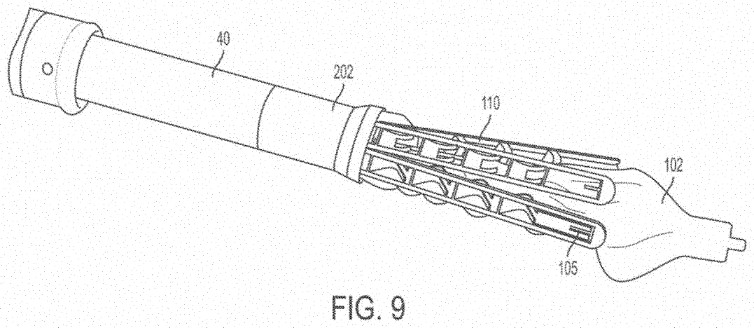

FIG. 9 illustrates an example implementation of a catheter assembly 40 attached to an implantable void-filling device 100 suitable for use with the void-filling devices shown in FIGS. 1-3. The system in FIG. 9 includes a catheter assembly 40 and an implantable void-filling implant 100. The void-filling implant 100 includes a connection hub 202, an inflatable implant 102, a coating 120, a plurality of independent anchors 110, and a plurality of independent tines 105 arranged along the length of each respective anchor 110. Prior to implantation, the independent anchors 110 are arranged in a close, straight configuration. After delivery and implantation of the void-filling implant, the independent anchors assume an expanded configuration. In some implementations, the independent anchors assume an expanded configuration upon exiting the catheter assembly 40 whereby the independent anchors curve outwardly upon exposure to an activation energy. In some implementations, prior to delivery, the independent anchors 110 are in a compressed state within the catheter assembly 40, after delivery the independent anchors assume an expanded configuration upon exiting the catheter assembly whereby the anchors are released from a compressed state.

FIG. 9 also includes an inflatable implant 102. Prior to delivery, the inflatable implant 102 remains in a deflated state. In some implementation, after implantation, an inflation material fills the inflatable implant 102 to a substantially zero pressure through an inner shaft contained within the catheter assembly 40. As mentioned above, in some implementations, the inflatable implant can include an ultra-compliant balloon configured to conform to the individual shape of the body cavity. In other implementations, the inflatable implant includes an over-sized balloon configured to conform to the individual shape of the body cavity, or a higher-pressure filled balloon held in place with the plurality of independent anchors 110 and the coating 120.

In some implementations, the length of the inflatable implant at its zero-pressure state may be about 12 mm to 24 mm, the diameter may be about 6 mm-18 mm, and the thickness of the inflatable implant may be about 50 microns or less to fit into a 12 Fr catheter.

FIG. 10 illustrates an example implementation of a connection assembly 200 between a catheter assembly 40 and an implantable void-filling device 100. The catheter assembly 40 in FIG. 10 includes an inner shaft 44, an outer shaft 42, and a connection assembly 200. The inner shaft 44 is contained within a lumen of the outer shaft 42 and is moveable relative to the outer shaft 42. The movability of the inner shaft 44 relative to the outer shaft can be axial, rotational, or both, and is controlled by a corresponding feature on the handle 20 on the catheter assembly 40, as discussed above in relation to FIG. 1. In some implementations, the inner shaft 44 defines the inflation lumen 46 that connects the inflation port (not shown) on the catheter assembly 40 to an interior of the inflatable implant 102. The inflation lumen (also referred to as “balloon fill lumen”) 46 is sized to accommodate the selected inflation fluid such that excessive force is not required to pass the inflation fluid through the lumen 46. Alternatively in some implementations, the lumen of the outer shaft may be used as an inflation lumen, depending on the configuration of the connection assembly 200.

The catheter shafts 42 and 44 terminate at a distal end and interact with, or become integral parts of, the connection assembly 200, described below. In at least some implementations, it is advantageous to provide rigid or semi-rigid materials at the distal ends of one or both shafts 42 and 44.

The connection assembly 200 joins the implantable void-filling device 100 to the catheter assembly 40 and establishes fluid communication between the inflation lumen 46 and the interior of the inflatable implant 102. The connection assembly 200 generally includes a connection hub 202, which is attached to and remains with the void-filling implant 100, and a locking hub 250, which is attached to and remains with the distal end of the catheter assembly 40. The connection hub 202 defines a hollow passage that leads to the interior of the inflatable implant 102. The connection hub 202 and the locking hub 250 are releasably attached to each other and together include the distal component of the detachment system.

Within the hollow passage of the connection hub 202 is contained a septum 204. The septum 204 defines an access lumen 206 that passes through the septum 204 and leads to the interior of the inflatable implant 102. The septum 204 is constructed and arranged such that the access lumen 206 accepts the inner shaft 44 of the catheter assembly 40 when the inner shaft 44 is inserted therethrough or at its junction. However, when the inner shaft 44 is removed from the septum 204, the access lumen 206 closes, or substantially closes, such that fluid contained within the interior of the inflatable implant 102 is not allowed to escape.

In one implementation, the septum 204 is made of a soft elastomer. The access lumen 206 is formed by making a small, expandable hole, a single slit, or multiple intersecting slits that expand or spread to accommodate the inner shaft 44 but substantially close when removed. In another implementation, the septum 204 includes a thick liquid plug. The thick liquid plug is formed from a very thick, almost wax-like liquid, which seals itself when the inner shaft 44 is removed.

In another implementation, the septum 204 includes a valve or flap at an end, preferably the distal end, thereof. The valve may include a simple hinged elastomer flap that is easily pushed open when the inner shaft 44 is inserted through the septum 204 and reseats itself over the access lumen 206 when the inner shaft 44 is removed.

At a proximal end of the connection hub 202 is a surface or rim 210. The rim 210 provides a surface against which a corresponding surface of the catheter assembly 40 can act if the device employs an axially actuated release mechanism. For example, in one implementation, the catheter assembly 40 includes a pusher catheter that is advanceable relative to the outer sheath 42 and the inner shaft 44, such that it may be used to prevent proximal movement of the implant 100 while the inner shaft 44 is being retracted from the implant 100.

Another implementation uses the self-expanding independent anchors 110 to provide a surface for disengagement of the implant 100 from the catheter assembly 40. The anchors 110 extend from the connection hub 202 as shown in FIGS. 1-3. Once the rigid anchors 110 expand, and the physician desires to disengage the implant 100, the outer sheath 42 is advanced until the distal end of the outer sheath 42 impinges against the expanded anchors 110. Enough resistance is provided by the expanded anchors 110 to allow retraction of the inner shaft 44 through the access lumen 206 of the septum 204 without causing proximal retraction of the implant 100.

In another implementation, the locking hub 250 is rotatably connected to the connection hub 202 with a mechanism such as a luer lock engagement or the like. The locking hub 250 may include a thread that is configured to mate with an interior surface of the connection hub 202. This implementation further includes a torque shaft 254, in place of or in addition to a pusher catheter, designed to transmit rotational force without twisting. Referring to FIG. 1, rotation of the release mechanism of the handle 22 transmits rotation energy through the torque shaft 254 to the locking hub 250. Resistance to rotation of the implant may be provided by advancing the outer sheath 42 and holding it against the implant 100, or by interference between the implant and the target site, or both. Because the implant 100 is resistant to rotation, the overall effect of rotating the release mechanism is a relative rotation between the locking hub 250 and the connection hub 202, resulting in a disengagement of the thread and a separation of the implant 100 from the catheter assembly.

Other methods of disengaging or releasing the implant 100 from the catheter assembly 40 include, but are not limited to, expansion of a linkage, chemical degradation, activation of heating elements resulting in a breakage of a tether, magnetic mechanisms, and the like.

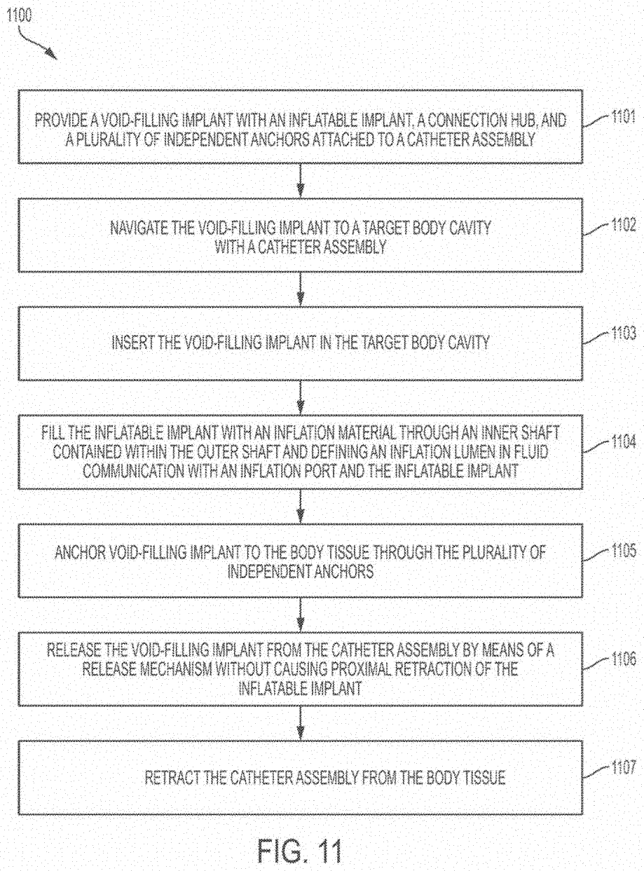

FIG. 11 illustrates an example method 1100 for delivering the implantable void-filling device into a subject's body cavity. Referring to FIGS. 1-10, the method includes providing an implantable void-filling device 100 with an inflatable implant 102, a connection hub 202, and a plurality of independent anchors 110 attached to a catheter assembly 40 (step 1101). Next, method 1100 includes navigating the implantable void-filling device 100 to a target body cavity with the catheter assembly 40 (step 1102). Next, method 1100 includes inserting the void-filling implant 100 in the target body cavity (step 1103). Method 1100 also includes filling the inflatable implant 102 with an inflation material through an inner shaft 44 contained within the catheter assembly 40 and defining an inflation lumen in fluid communication with an inflation port and the inflatable implant 102 (step 1104). Next, method 1100 includes anchoring the void-filling implant to body tissue with the plurality of independent anchors 110 (step 1105). Next, method 1100 includes releasing the void-filling implant 100 from the catheter assembly 40 by means of a release mechanism without causing proximal retraction of the implant 100 (step 1106). Method 1100 also includes retracting the catheter assembly 40 from the subject (step 1107).

As mentions above, first method 1100 includes providing a void-filling implant 100 that includes an inflatable implant 102, a connection hub 202, and a plurality of independent anchors 110, the void-filling implant being attached to a catheter assembly 40 (step 1101). The plurality of independent anchors 110 include a series of tines 105 arranged along the length of each independent anchor 110. In some implementations, the series of tines may include between two or twelve tines 105 per independent anchor. In some implementations, the series of tines 105 may exceed twelve tines 105 per independent anchor 110.

Next, method 1100 includes navigating the void-filling implant 100 to a target body cavity by means of a catheter assembly 40 (step 1102). In some implementations, the void-filling implant 100 may be navigated to the subject's body cavity with the help of fluoroscopy and intracardiac echocardiography. In some implementations, the void-filling may be navigated to the subject's body cavity with the help of ultrasound. Method 1100 also includes inserting the void-filling implant 100 within the body cavity (step 1103).

Next, method 1100 includes filling the inflatable implant 102 with an inflation material through an inner shaft 44 contained within the catheter assembly 40, which defines an inflation lumen in fluid communication with an inflation port and the inflatable implant (step 1104). Non-limiting examples of the inflation fluid may include liquid-based substances such as saline, iodinated or gadolinium-based contrast agents, curable polymers, curable hydrogels, silicon or foam. In some implementations, the inflation fluid may include materials capable of phase change. The phase change may be induced by gradual chemical reactions to a stimulus-triggered event. Examples of such events include exposure to a predetermined pH, electrochemical activation, ultrasonically induced mixing, optical methods, or inductive methods. As mentioned above, the inflatable implant may be filled with two or more substances simultaneously or sequentially. In some implementations, the inflatable implant 102 may also include a rate-based delivery where the implant is filled based on the known volume of the body cavity and inflation is conducted in a controlled fashion by quantitative or semi-quantitative volume based on pre-procedural or real-time 2-dimensional or 3-dimensional imaging. Also, the void-filling implant may include image-based filling such that real-time imaging monitors the size of the implant.

Next, method 1100 includes anchoring the void-filling implant 100 to the subject's body tissue by means of the plurality of independent anchors 110 (step 1105). As mentioned above, each independent anchor 110 includes a series of tines 105 attached to the length of the anchor 110. The tines 105 can act as barbs, wherein after the anchors 110 expand after delivery, the tines 105 also unfold into an engaged position in order to fully attach to the subject's body tissue. In some implementations, the tines 105 are made from a material, such as nitinol, that is sufficiently hard so to pierce the body tissue and prevent migration of the void-filling implant 100. In some implementations, the number of tines 105 along each anchor 110 leg creates enough friction against the body tissue to prevent migration of the implant without piecing the body tissue.

Method 1100 also includes releasing the void-filling implant from the catheter assembly 40 by means of a release mechanism without causing proximal retraction of the implant 100 (step 1107). As mentioned above in FIG. 10 one implementation of the release mechanism to release the void-filling implant from the catheter assembly may include the connection hub 202 engaged with a locking hub 250 such that a predetermined relative motion between the connection hub 202 and the locking hub 250 results in disengagement between the catheter assembly 40 and the void-filling implant 100. In some implementations, the predetermined relative motion includes a relative rotation between the connection hub 202 and the locking hub 250, wherein the connection hub 202 and the locking hub 250 connect and disengage. For example, the connection hub 202 and the locking hub 250 may be mated similar to mating found in Chicago coupling or the mating found in Storz and Pin coupling. In other implementations, the predetermined relative motion may include a relative axial motion between the connection hub 202 and the locking hub 250. Next, method 1100 includes retracting the catheter assembly 40 from the subject's body while the void-filling implant remains within the body cavity (step 1107).

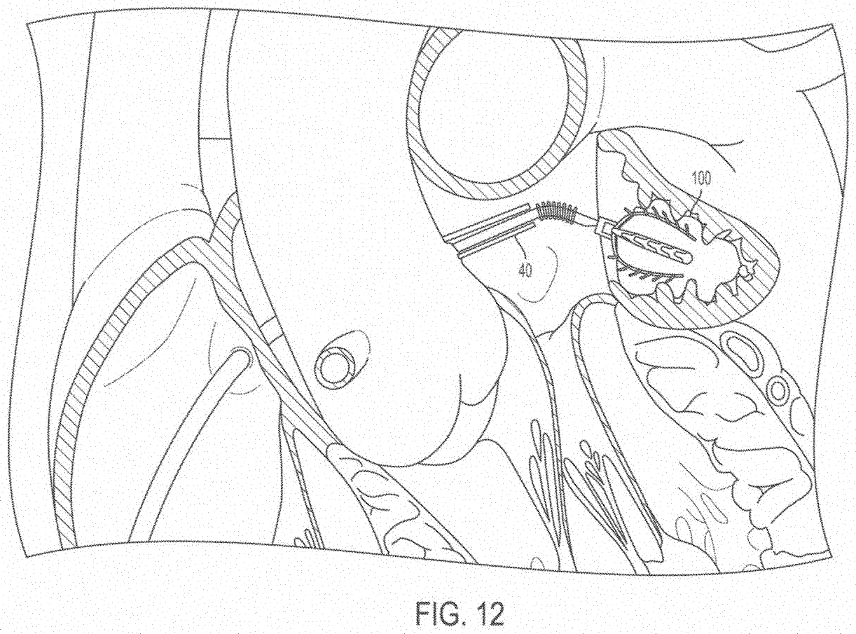

FIG. 12 illustrates an example implementation of a catheter assembly 40 delivering an implantable void-filling device 100 to a LAA. Similar to FIG. 11 above, in FIG. 12, the catheter assembly 40 first navigates the void-filling implant 100 to the LAA in the left atrium of the heart. Next, the catheter assembly inserts the void-filling implant in the LAA and fills the inflatable implant with an inflation material through an inner shaft contained within the catheter assembly. In some implementations, the inflation material includes a plurality of fluids, gases, or curing liquids that interact with each other. Next, as shown in FIG. 12, a plurality of independent anchors 110 assume an expanded configuration after delivery, which anchors the implant 100 within the body cavity. In some implementations, the implant 100 also includes a coating (not shown) that prevents the balloon from expanding into the left atrium. The coating can also enhance endothelialization of the implant to the body tissue to reduce the risk of thrombosis. Next, the catheter assembly 40 releases the inflatable implant. In some implementations, the void-filling implant 100 can be used to fill defective body cavities other than the LAA, such as septal defects, aneurysms, pseudo-aneurysms, or colonic outpouchings.

Although the invention has been described in terms of particular implementations and applications, one of ordinary skill in the art, in light of this teaching, can generate additional implementations and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A system for occluding a body cavity, comprising:
an implantable void-filling device comprising:
an inflatable implant defining an interior of the implantable void-filling device, wherein the inflatable implant is capable of being filled with an inflation material to cause the inflatable implant to expand from a collapsed configuration to an expanded configuration;
a connection hub attached to an exterior surface of the inflatable implant; and,
a plurality of independent anchors coupled to and extending out from the connection hub along the length of the inflatable implant, such that the plurality of anchors collectively surround the inflatable implant, and the connection hub and anchors are configured such that expansion of the anchors away from the surface of the inflatable implant anchors the void filling device to a body tissue.

2. The system of claim 1, wherein the plurality of anchors comprise tines arranged along the lengths of the respective anchors configured to, upon exposure to an activation energy, curve outward from the respective anchors to engage a body tissue.

3. The system of claim 1, further comprising a coating configured to cover the connection hub and at least a portion of the lengths of the anchors.

4. The system of claim 1, wherein the inflation material comprises a gas, a liquid, or a gel.

5. The system of claim 1, wherein the plurality of anchors comprise of a shape-memory alloy.

6. The system of claim 1, wherein the inflatable implant comprises an ultra-compliant balloon configured to conform to the individual shape of the body cavity.

7. The system of claim 6, wherein the ultra-compliant balloon comprises of an elastomer, a polymer, or a fabric reinforced polymer.

8. The system of claim 1, wherein the inflatable implant comprises an over-sized balloon configured to conform to the individual shape of the body cavity.

9. The system of claim 1, wherein the inflatable implant is configured to conform to the individual shape of the body cavity at substantially zero pressure.

10. The system of claim 1, wherein the coating comprises a fabric covering attached to the connection hub and at least a portion of the independent anchors.

11. The system of claim 1, further comprising a catheter assembly configured to deliver the implantable void-filling device to the body cavity comprising:
a handle including an inflation port attachable to an inflation fluid cartridge containing inflation fluid; and,
an inner shaft slideably contained within an outer shaft and defining an inflation lumen in fluid communication with the inflation port and the inflatable implant, wherein a connection assembly removably joins the inflatable implant and the catheter assembly.

12. The system of claim 11, further comprising a release mechanism configured to release the inner shaft of the catheter assembly through the access lumen of the septum without causing proximal retraction of the inflatable implant.

13. The system of claim 11, wherein the curved anchors have a straightened delivery configuration and an expanded configuration, wherein the anchors assume the expanded configuration upon exiting the outer shaft whereby the anchors passively or actively curve outwardly upon exposure to an activation energy.

14. The system of claim 11, wherein the curved anchors have a straightened delivery configuration and an expanded configuration, wherein the anchors assume the expanded configuration upon exiting the outer shaft whereby the anchors release from a compressed state.

15. A method for occluding a body cavity, comprising:
providing an implantable void-filling device comprising:
an inflatable implant defining an interior of the implantable void-filling device, wherein the inflatable implant is capable of being filled with an inflation material to cause the inflatable implant to expand from a collapsed configuration to an expanded configuration;
a connection hub attached to an exterior surface of the inflatable implant;
a plurality of independent anchors extending coupled to and extending out from the connection hub along the length of the inflatable implant, such that the plurality of anchors collectively surround the inflatable implant, and the connection hub and anchors are configured such that expansion of the anchors away from the surface of the inflatable implant anchors the void filling device to a body tissue; and,
providing a catheter assembly configured to deliver the implantable void-filling device to the body cavity, comprising:
navigating the implantable void-filling device to a target body cavity with the catheter assembly,
inserting the implantable void-filling device in the target body cavity;
filling the inflatable implant with an inflation material through an inner shaft contained within the catheter assembly;
anchoring the implantable void-filling device to the inside of the body cavity through the plurality of independent anchors;
releasing the inflatable implant from the catheter assembly; and,
retracting the delivery device from the body tissue.

16. The method of claim 15, wherein navigating an inflatable implant to a target body cavity with the catheter assembly comprises navigating the catheter assembly having said inflatable implant stored in a distal end thereof.

17. The method of claim 15, wherein inserting the inflatable implant in the target body cavity comprises ejecting the inflatable implant from within a distal end of the catheter assembly.

18. The method of claim 15, wherein filling the inflatable implant with an inflation material comprises filling the inflatable implant with a plurality of fluids, gases, or curing liquids that interact with each other.

19. The method of claim 15, wherein releasing the inflatable implant from the catheter assembly comprises rotating a component of the catheter assembly relative to a mating component of the void-filling device, such that the two components disengage each other.

20. The method of claim 15, wherein anchoring the implantable void-filling device to the inside of the body cavity comprises engaging the body tissue with tines arranged along the lengths of the respective anchors configured to, upon exposure to an activation energy, curve outward from the respective anchors to engage a body tissue.

21. The method of claim 15, wherein the plurality of anchors comprise a shape memory alloy nitinol.

22. The method of claim 18, comprising inflating the inflatable implant wherein the inflatable implant comprises an ultra-compliant balloon configured to conform to the individual shape of the body cavity.

23. The method of claim 22, wherein the ultra-compliant balloon comprises of an elastomer, a polymer, or a fabric reinforced polymer.
